SEQUENCE LISTING (39 sequences)

SEQ ID NO: 1 - protein, 8 aa, Artificial
  Vascular endothelial cell-targeting peptide - P1
CSLRSPPS

SEQ ID NO: 2 - protein, 8 aa, Artificial
  Vascular endothelial cell-targeting peptide - P3
CNNSGMRN

SEQ ID NO: 3 - DNA, 23 bp, Artificial
  bGHpA primer - F
tctagttgccagccatctgttgt

SEQ ID NO: 4 - DNA, 17 bp, Artificial
  bGHpA primer - R
tgggagtggcaccttca

SEQ ID NO: 5 - DNA, 20 bp, Artificial
  Cre primer - F
agaggaaagtctccaacctg

SEQ ID NO: 6 - DNA, 20 bp, Artificial
  Cre primer - R
acacagacaggagcatcttc

SEQ ID NO: 7 - DNA, 21 bp, Artificial
  eGFP primer - F
gccacaacgtctatatcatgg

SEQ ID NO: 8 - DNA, 20 bp, Artificial
  eGFP primer - R
ggtgttctgctggtagtggt

SEQ ID NO: 9 - DNA, 22 bp, Artificial
  M13 phage primer - F
ttattcgcaattcctttagtgg

SEQ ID NO: 10 - DNA, 20 bp, Artificial
  M13 phage primer - R
ccctcatagttagcgtaacg

SEQ ID NO: 11 - protein, 7 aa, Artificial
  U4 phage - Vascular endothelial cell targeting peptide
NCLANPC

SEQ ID NO: 12 - protein, 9 aa, Artificial
  U5 phage - Vascular endothelial cell targeting peptide
CSVTKSTYC

SEQ ID NO: 13 - protein, 9 aa, Artificial
  U1 phage - Vascular endothelial cell targeting peptide
CRSVSNNNC

SEQ ID NO: 14 - protein, 9 aa, Artificial
  U2 phage - Vascular endothelial cell targeting peptide
CSHMNESMC

SEQ ID NO: 15 - protein, 9 aa, Artificial
  U3 phage - Vascular endothelial cell targeting peptide
CRFHMRETC

SEQ ID NO: 16 - protein, 9 aa, Artificial
  M1 phage - Vascular endothelial cell targeting peptide
CLYLGAEMC

SEQ ID NO: 17 - protein, 9 aa, Artificial
  M3 phage - Vascular endothelial cell targeting peptide
CTITGQGAC

SEQ ID NO: 18 - protein, 9 aa, Artificial
  M2 phage - Vascular endothelial cell targeting peptide
CTQSGVRNC

SEQ ID NO: 19 - protein, 9 aa, Artificial
  M7 phage - Vascular endothelial cell targeting peptide
CTQSGVRNC

SEQ ID NO: 20 - protein, 6 aa, Artificial
  M4 phage - Vascular endothelial cell targeting peptide
VGGWNC

SEQ ID NO: 21 - protein, 9 aa, Artificial
  M5 phage - Vascular endothelial cell targeting peptide
CILSNNFFC

SEQ ID NO: 22 - protein, 9 aa, Artificial
  M6 phage - Vascular endothelial cell targeting peptide
CMVSMILRC

SEQ ID NO: 23 - DNA, 27 bp, Artificial
  U4 phage - Vascular endothelial cell targeting peptide
tgttgaaattgtttagcaaatccctgc

SEQ ID NO: 24 - DNA, 27 bp, Artificial
  U5 phage - Vascular endothelial cell targeting peptide
tgttctgtgacgaagtcgacgtattgc

SEQ ID NO: 25 - DNA, 27 bp, Artificial
  U1 phage - Vascular endothelial cell targeting peptide
tgtcgtagtgtgtcgaataataattgc

SEQ ID NO: 26 - DNA, 27 bp, Artificial
  U2 phage - Vascular endothelial cell targeting peptide
tgttctcatatgaatgagtcgatgtgc

SEQ ID NO: 27 - DNA, 27 bp, Artificial
  U3 phage - Vascular endothelial cell targeting peptide
tgtagatttcatatgagggagacttgc

SEQ ID NO: 28 - DNA, 27 bp, Artificial
  M1 phage - Vascular endothelial cell targeting peptide
tgtctgtatttgggtgctgagatgtgc

SEQ ID NO: 29 - DNA, 27 bp, Artificial
  M3 phage - Vascular endothelial cell targeting peptide
tgtacgattactggtcagggtgcttgc

SEQ ID NO: 30 - DNA, 27 bp, Artificial
  M2 phage - Vascular endothelial cell targeting peptide
tgtacgcagtcgggggttaggaattgc

SEQ ID NO: 31 - DNA, 27 bp, Artificial
  M7 phage - Vascular endothelial cell targeting peptide
tgtacgcagtcgggggttaggaattgc

SEQ ID NO: 32 - DNA, 27 bp, Artificial
  M4 phage - Vascular endothelial cell targeting peptide
tgtaactgagtcgggggttggaattgc

SEQ ID NO: 33 - DNA, 27 bp, Artificial
  M5 phage - Vascular endothelial cell targeting peptide
tgtatccttagtaataatttcttttgc

SEQ ID NO: 34 - DNA, 27 bp, Artificial
  M6 phage - Vascular endothelial cell targeting peptide
tgtatggtaagtatgatcctacggtgc

SEQ ID NO: 35 - DNA, 15420 bp, Artificial
  Ad pAdDeltaF6 helper plasmid
tcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatccccgggtactctagttaattaactagaggatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctg

SEQ ID NO: 36 - DNA, 7468 bp, Artificial
  pAAV2/2 rep2/cap2 plasmid
acctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgccgatatcgttaacgccccgcgccggccgctctagaactagtggatcccccggaagatcagaagttcctattccgaagttcctattctctagaaagtataggaacttctgatctattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggagagtcctgtattagaggtcacgtgagtgttttgcgacattttgcgacaccatgtggtcacgctgggtatttaagcccgagtgagcacgcagggtctccattttgagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacg

SEQ ID NO: 37 - DNA, 5504 bp, Artificial
  pAAV-CMV-eGFP Transfer plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 38 - DNA, 7330 bp, Artificial
  pAAV2/9n rep2/cap9 plasmid
gtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgg

SEQ ID NO: 39 - DNA, 6422 bp, Artificial
  pAAV-CMV-Cre Transfer plasmid
ctgcgcgctcgctcgctcactgaggccgcccgggcaaagcccgggcgtcgggcgacctttggtcgcccggcctcagtgagcgagcgagcgcgcagagagggagtggaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcgggcccgggatccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactgatgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcc

(19) United States
(12) Patent Application Publication
(10) Pub. No.: US 2025/0325703 A1
(43) Pub. Date: Oct. 23, 2025

(54) COMPOSITION FOR DELIVERING GENE TO BRAIN TISSUE AND USE THEREOF

(71) Applicant: Seung Min LEE, Yongin-si, Gyeonggi-do (KR)

(72) Inventor: Seung Min LEE, Yongin-si, Gyeonggi-do (KR)

(21) Appl. No.: 18/031,944

(22) PCT Filed: Oct. 14, 2021
(86) PCT No.: PCT/KR2021/014243

§ 371 Date: Apr. 14, 2023

(30) Foreign Application Priority Data
Oct. 15, 2020 (KR) 10-2020-0133737

(51) Int. Cl. A61K 48/00; C12N 15/86

(52) U.S. Cl. CPC A61K 48/0058; A61K 48/0041; C12N 15/86; C12N 2750/14143; C12N 2750/14145

(57) ABSTRACT

Provided is a composition including a recombinant viral vector, a dendrimer, and a cerebrovascular cell-targeting peptide, for gene delivery to brain tissue. According to the composition, target genetic material may be delivered to cerebrovascular cells, or to brain tissue through the blood brain barrier (BBB), and due to this, a brain disease may be prevented or treated.

### CROSS-REFERENCE TO RELATED APPLICATIONS

This application is a U.S. national phase application filed under 35 U.S.C. § 371 of International Application Number PCT/KR2021/014243 filed Oct. 14, 2021, designating the United States, which claims priority from Korean Application Number 10-2020-0133737, filed Oct. 15, 2020.

### REFERENCE TO A SEQUENCE LISTING

The Sequence Listing Associated with this application is filed in electronic format via EFS-Web and is hereby incorporated into the specification in its entirety. The name of the text file containing the Sequence Listing is “157381_SequenceListing”. The size of the text file is 60 KB, and the file was created on Nov. 17, 2023.

### TECHNICAL FIELD

The present application relates to a composition for delivering a gene to brain tissue, and a use thereof.

### BACKGROUND ART

Gene therapy aims to correct linkage genes that underlie the development of a disease. A common approach to addressing this challenge involves transferring normal genes into the nucleus. Delivery of a corrective gene to target cells of a subject may be accomplished by using many methods, including methods using viral vectors. Among the many viral vectors available (for example, retroviruses, lentiviruses, adenoviruses, etc.), adeno-associated viruses (AAVs) are gaining popularity as versatile vectors in gene therapy.

Viral vectors have a number of advantages over plasmid DNA with regard to delivery of genetic material. For example, expression of a heterologous gene from a plasmid is short-lived, plasmids are generally larger in size, plasmids need to be physically engineered to be delivered into cells, and transfer of a gene such as dystrophin by a plasmid may trigger an immune response in the host and is associated with low gene delivery efficiency. On the other hand, since viral vectors can efficiently infect various cells, have high gene delivery efficiency, and may be produced at high titers, and therefore, viral vectors may be used as a very suitable vector system for gene therapy as long as the viral vectors do not induce an immune response.

On the other hand, research on gene therapy using viral vectors is ongoing for various diseases occurring in the brain, and Korean Patent Publication No. 10-2010-0124090 discloses a pharmaceutical composition for preventing or treating a brain disease including a recombinant virus including a human arginine decarboxylase (ADC) gene.

However, most of these existing technologies are technologies for regulating intracellular genes by an ex vivo method of transfecting cells isolated from living organisms with viral vectors ex vivo, and researches on in vivo gene therapy by administering recombinant viruses directly to patients to treat a brain disease are insufficient. This is related to the issue that a viral vector, which is a carrier, must pass through cerebrovascular cells or a blood-brain barrier (BBB) in order to transfer a gene to brain tissue.

With this background, the present inventors developed a virus-dendrimer-peptide complex including: a recombinant viral vector; and a dendrimer and a cerebrovascular cell-targeting peptide connected to a surface of the recombinant virus, and by confirming the use of the complex for gene delivery to brain tissue, completed the present application.

### DESCRIPTION OF EMBODIMENTS

### Technical Problem

An object of the present application is to provide a composition for delivering a gene to brain tissue, including a virus-dendrimer-peptide complex including: a recombinant viral vector; and a dendrimer and a cerebrovascular cell-targeting peptide connected to a surface of the recombinant virus.

Another object of the present application is to provide a pharmaceutical composition for preventing or treating a brain disease including the composition as an active ingredient.

Still another object of the present application is to provide a method of preparing the virus-dendrimer-peptide complex for delivering a gene to brain tissue, including connecting the dendrimer and the cerebrovascular cell-targeting peptide to a surface of the recombinant viral vector.

Still another object of the present application is to provide a use of the virus-dendrimer-peptide complex including the recombinant viral vector, and the dendrimer and the cerebrovascular cell-targeting peptide connected to the surface of the recombinant virus, for delivering a gene to brain tissue.

### Solution to Problem

Each description and embodiment disclosed in the application may also be applied to other descriptions and embodiments. That is, all combinations of various elements disclosed in the application fall within the scope of the application. In addition, it should not be construed that the scope of the present application is limited by the detailed description described below. In addition, those skilled in the art may be able to recognize, or confirm many equivalents to specific aspects of the present application described herein by using only experiments commonly used in the art. Such equivalents are intended to be included in this application.

An aspect provides a composition for delivering a gene to brain tissue, including a virus-dendrimer-peptide complex including: a recombinant viral vector; and a dendrimer and a cerebrovascular cell-targeting peptide connected to a surface of the recombinant virus.

The term “viral vector”, used herein, refers to a carrier developed by using a virus to inject genetic material such as DNA or RNA into cells or living organisms.

The term “recombination”, used herein, refers to a nucleic acid, vector, polypeptide, or protein that has been produced by using a DNA recombination (cloning) method and that may be distinguished from native or wild-type nucleic acids, vectors, polypeptides, or proteins.

The recombinant viral vector includes any recombinant virus, and specifically, any virus that may be included in a therapeutic agent, vaccine, drug delivery system, vector, or gene delivery system and used to treat a disease may be included.

Specifically, the recombinant viral vector includes, for example, an adenoviral vector, an adeno-associated viral vector, a vaccinia viral vector, a lentiviral vector, a retroviral vector, a baculoviral vector, or a herpes simplex viral vector, but is not limited thereto.

The recombinant viral vector may most preferably be an adeno-associated viral vector.

The term “adeno-associated virus (AAV)”, used herein, refers to a single-chain DNA virus, which is a helper vector-dependent human parvovirus.

A genome size of the adeno-associated virus may be about 4.6 kbp, the N-terminus portion of the genome may encode a rep gene involved in viral replication and viral gene expression, and the C-terminus portion may encode a cap gene that encodes a viral capsid protein, and inverted terminus repeats (ITRs) of about 145 bases inserted may be included at both terminuses. For example, about four proteins are translated from the rep region, which are classified as rep78, rep68, rep52, and rep40 according to their molecular weight, and perform an important function in DNA replication of AAV. In addition, about three proteins, namely VP1, VP2, and VP3, are translated from the cap region, and these are structural proteins required for particle formation (viral assembly) of AAV.

Since AAV is capable of infecting non-dividing cells and has an ability to infect various types of cells, AAV may be suitable as a gene delivery system of the present disclosure. Detailed descriptions of preparation and use of AAV vectors are disclosed in detail in U.S. Pat. Nos. 5,139,941 and 4,797,368.

The AAV vector may be recombinant adeno-associated virus (rAAV) vector.

The AAV vector may be rAAV1, rAAV2, rAAV3, rAAV4, rAAV5, rAAV6, rAAV7, rAAV8, rAAV9, rAAV10, rAAV11, rAAV12, rAAV2/1, rAAV2/2, rAAV2/3, rAAV2/4, rAAV2/5, rAAV2/6, rAAV2/7, rAAV2/8, rAAV2/9, or a homolog or variant thereof.

The recombinant viral vector may include a target gene. Specifically, the target gene may be a therapeutic gene capable of treating a brain disease by being delivered to brain tissue.

The therapeutic gene may refer to a gene (polynucleotide sequence) that may encode a polypeptide that may exhibit a therapeutic or preventive effect when expressed in a cell.

The therapeutic gene is not limited by a type of a target disease as long as the gene may be included in the recombinant viral vector, and may include a separate promoter for gene expression. In addition, the therapeutic gene may be included singly or in combination of two or more.

A form in which the therapeutic gene is included in the recombinant viral vector is not limited, and for example, the therapeutic gene may be a virus that has a therapeutic effect by itself or modified to have a therapeutic effect, or the therapeutic gene may be included in a form of being bound to or carried in the recombinant viral vector, but is not limited thereto.

The term “dendrimer”, used herein, refers to a molecule in which chains of molecules spread three-dimensionally from the center to the outside according to a certain rule, and refers to a branch-shaped macromolecule in which a branch-shaped unit structure repeatedly extends from the core.

The dendrimer may simultaneously have molecularity of a low molecule or supramolecule and materiality of a polymer, and therefore, the dendrimer may be defined as a macromolecular compound having duality (molecularity and materiality) between a polymer and a supramolecule.

The dendrimers may be a dendrimer of any one type selected from the group consisting of a polyamidoamine dendrimer, polylysine dendrimer, polyimine dendrimer, polypropyleneimine dendrimer, polyester dendrimer, polyether dendrimer, polyglutamic acid dendrimer, polyaspartic acid dendrimer, polyglycerol dendrimer, and polymelamine dendrimer, or a dendrimer composed of a copolymer of two or more types selected from the group.

The dendrimer may be most preferably a polyamidoamine (PAMAM) dendrimer.

The PAMAM dendrimer may be a spherical nanoparticle having a diameter of about 1 nm to about 13 nm from generation 0 to generation 10 and may have a diameter increasing by about 1 nm per generation.

In an embodiment, for the PAMAM dendrimer, a 0 generation PAMAM dendrimer having 4 surface amine groups and a molecular weight of about 480 Da to about 550 Da is defined as PAMAM dendrimer G0, a 1st generation PAMAM dendrimer having 8 surface amine groups and a molecular weight of about 1,200 Da to about 1,700 Da is defined as PAMAM dendrimer G1, a 2nd generation PAMAM dendrimer having 16 surface amine groups and a molecular weight of about 3,000 Da to about 3,500 Da is defined as PAMAM dendrimer G2, a 3rd generation PAMAM dendrimer having 32 surface amine groups and a molecular weight of about 6,800 Da to about 7,300 Da is defined as PAMAM dendrimer G3, a 4th generation PAMAM dendrimer having 64 surface amine groups and a molecular weight of about 13,500 Da to about 15,000 Da is defined as PAMAM dendrimer G4, and a 5th generation PAMAM dendrimer having 128 surface amine groups and a molecular weight of about 27,000 Da to about 30,000 Da is defined as PAMAM dendrimer G5.

In an embodiment, the dendrimer may be a PAMAM dendrimer G1, a PAMAM dendrimer G2, a PAMAM dendrimer G3, a PAMAM dendrimer G4, a PAMAM dendrimer G5, a PAMAM dendrimer G6, a PAMAM dendrimer G7, a PAMAM dendrimer G8, a PAMAM dendrimer G9, or a PAMAM dendrimer G10, and most preferably, PAMAM dendrimer G2 or PAMAM dendrimer G5.

A molecular structure of the PAMAM dendrimer may vary, specifically, a core of the PAMAM dendrimer may be selected from among 5 core types (cystamine, diaminobutane, diaminohexane, diamonododecane, and ethylenediamine), and a functional group selected from nine surface functional groups (amine, amidoethylethanolamine, amidoethanol, sodium carboxylate, succinamic acid, hexylamide, carbomethoxypyrrolidinone, tris-hydroxymethyl-amidomethane, and poly-ethyleneglycol) may be bound to a surface of the PAMAM dendrimer.

The dendrimer may exhibit various external charge patterns. Specifically, the dendrimer may have a positively charged amino-terminus (cation), a neutral hydroxyl-terminus (neutral), or a negatively charged carboxyl-terminus (anion) provided by surface molecules at the end of the outermost surface.

In an embodiment, the end of the outermost surface of the dendrimer may be bound to an amine group (—NH₂).

In an embodiment, the dendrimer may have a positive charge at the end of the outermost surface. Specifically, the dendrimer may have a positively charged amino terminus (cation) at the end of the outermost surface.

Therefore, when a surface of the recombinant viral vector is negatively charged, an electrostatic interaction may occur between the surface of the recombinant viral vector and the surface of the dendrimer due to the positive surface charge of the dendrimer, and thereby, the dendrimer may be easily coated on the surface of the recombinant viral vector. Specifically, the dendrimer may be strongly bound and coated on the surface of the recombinant viral vector in a short time by the electrostatic interaction.

Since vascular endothelium contains highly polar glycosaminoglycan (GAG) on its surface, and GAG includes heparin which has a high negative charge density, the positively charged surface of the dendrimer and the negatively charged surface of the vascular endothelium may cause electrostatic interaction.

One end of the dendrimer may be connected to the cerebrovascular cell-targeting peptide by a linker.

The linker may include polyethylene glycol (PEG), but is not limited thereto, and any linker may be used as long as the linker may connect the dendrimer and the cerebrovascular cell-targeting peptide.

In an embodiment, the cerebrovascular cell-targeting peptide may be a peptide including an amino acid sequence of SEQ ID NO: 1 or SEQ ID NO: 2.

In an embodiment, the cerebrovascular cell-targeting peptide including the amino acid sequence of SEQ ID NO: 2 may have significantly improved targeting ability for cerebrovascular endothelial cells.

A complex in which the dendrimer and the cerebrovascular cell-targeting peptide are linked may be coated on the surface of the recombinant viral vector to form a virus-dendrimer-peptide complex.

The virus-dendrimer-peptide complex may deliver a gene to brain tissue.

The brain tissue may be cerebrovascular cells or cranial nerve cells. Specifically, the cerebrovascular cells may be cerebrovascular endothelial cells.

The virus-dendrimer-peptide complex may target brain tissue, such as cerebrovascular cells or cranial nerve cells, by means of the cerebrovascular cell-targeting peptide included therein.

In addition, the virus-dendrimer-peptide complex may pass through a brain barrier such as a cell membrane of brain tissue such as the cerebrovascular cells or cranial nerve cells or the blood-brain barrier, due to the positive surface charge of the dendrimer included therein.

The term “blood-brain barrier (BBB)”, used herein, refers to a functional structure surrounding the brain in order for the brain to function stably, which severely restricts passage of various biomolecules and ions between blood vessels and the brain. Elements constituting the blood-brain barrier include cerebrovascular endothelial cells, blood vessel basal membrane, and astrocytes surrounding blood vessels. In particular, cerebrovascular endothelial cells constituting the blood-brain barrier have highly developed tight junctions, fewer pinocytic vesicular transporters, and less fenestrations than endothelial cells in other regions in terms of morphology and structure, making it hard for materials to pass through well.

Nonetheless, according to an embodiment, the virus-dendrimer-peptide complex targets cerebrovascular cells (specifically, cerebrovascular endothelial cells) or cranial nerve cells, which are brain tissue, and passes through the cell membrane of the cells or crosses the blood-brain barrier, and thus, may deliver a gene to brain tissue such as cerebrovascular cells (specifically, cerebrovascular epithelial cells), or cranial nerve cells.

As described above, in order to transfer a gene to brain tissue, it is important that the virus-dendrimer-peptide complex passes through the cell membrane of brain tissue such as cerebrovascular cells (specifically, cerebrovascular endothelial cells) or cranial nerve cells or the blood-brain barrier.

Therefore, by optimizing a density of the positive charge of the dendrimer and an amount of the cerebrovascular cell-targeting peptide, the efficiency of passage through the cell membrane of brain tissue or the blood-brain barrier may be increased, thereby significantly improving the gene delivery effect into brain tissue.

For example, in the virus-dendrimer-peptide complex, when an amount of the cerebrovascular cell-targeting peptide linked to a surface of the dendrimer increases, the surface positive charge density of the virus-dendrimer-peptide complex may decrease. As a result, passage efficiency of the virus-dendrimer-peptide complex through the cell membrane of brain tissue or the blood-brain barrier may be reduced.

In addition, even when amounts of both the dendrimer and the cerebrovascular cell-targeting peptide are excessively increased in the virus-dendrimer-peptide complex, the molecular size of the virus-dendrimer-peptide complex increases, resulting in reduced passage efficiency across the cell membranes of brain tissue or the blood-brain barrier.

In addition, in the virus-dendrimer-peptide complex, when the amount of the cerebrovascular cell-targeting peptide linked to the surface of the dendrimer decreases, even when the surface positive charge density of the virus-dendrimer-peptide complex increases, targeting ability of the virus-dendrimer-peptide complex for brain tissue such as cerebrovascular cells or cranial nerve cells may be reduced.

The term “gene delivery (gene transfer)”, used herein, refers to a method or system for reliably inserting an external nucleic acid sequence, such as DNA, into a host cell. Such methods may result in transient expression of unintegrated transferred DNA, extrachromosomal replication and expression of the transferred replicon (for example, an episome), or integration of the transferred genetic material into genomic DNA of the host cell.

The composition for gene delivery to brain tissue including the virus-dendrimer-peptide complex may be prepared as a composition suitable for administration to a subject according to a method that may be easily performed by a person skilled in the art to which the present disclosure belongs. Specifically, the composition may include a pharmaceutically acceptable carrier. The pharmaceutically acceptable carrier includes any and all solvents, dispersion media, coatings, antibacterial and antifungal agents, isotonic and absorption delaying agents, and the like that are physiologically compatible.

Examples of such pharmaceutically acceptable carriers include one or more of water, saline solution, phosphate buffered saline, dextrose, glycerol, ethanol, and the like, as well as combinations thereof. In addition, the composition may be formulated by using a pharmaceutically acceptable carrier and/or excipient, and prepared in a unit dosage form, or prepared by putting the composition into a multi-dose container. In this regard, the formulation may be in a form of a solution, suspension, or emulsion in oil or aqueous medium, or may be in a form of an extract, powder, granule, tablet, or capsule, and may additionally include a dispersing agent or a stabilizer.

Another aspect provides a pharmaceutical composition for preventing or treating a brain disease, including the composition including the virus-dendrimer-peptide complex for gene delivery to brain tissue as an effective ingredient.

The term “effective ingredient” used herein, refers to an ingredient in an appropriately effective amount for bringing about a beneficial or desirable clinical or biochemical outcome. Specifically, the term may mean an effective amount of the virus-dendrimer-peptide complex.

The effective amount may be administered once or more times, and may be an appropriate amount for preventing a disease, alleviating symptoms, reducing an extent of the disease, stabilizing a disease state (that is, not worsening), delaying or reducing a rate of disease progression, improving, or transiently mitiating and ameliorating the disease state (partially or entirely), as non-limiting examples. The effective amount may be determined according to a type and severity of the disease of a subject, activity of the drug, sensitivity to the drug, administration time, route of administration and excretion rate, treatment period, concurrently used drugs, and other factors well known in the medical field.

The brain disease is a disease that occurs in the brain and may include any disease to which gene therapy may be applied.

The brain disease may be a cranial nerve disease or a cerebrovascular disease, but is not limited thereto. Specifically, the brain disease may be Lou Gehrig's disease, Alzheimer's disease, Parkinson's disease, Huntington's disease, stroke, traumatic brain injury, amyotrophic lateral sclerosis, spinal cord injury and myelitis, epilepsy, seizure-related disorders, acute brain injury, chronic brain injury, chronic headache, migraine, and diseases related to the chronic headache and the migraine, neurodegenerative disease, mild cognitive impairment, cerebral infarction, vascular dementia, frontotemporal dementia, Lewy body dementia, Creutzfeldt-Jakob disease, syphilis, acquired immunodeficiency syndrome, and other viral infections, brain abscess, sclerosis, metabolic disease-induced dementia, hypoxia, Pick's disease, attention deficit-hyperactivity disorder, schizophrenia, depression, bipolar disorder, post-traumatic stress disorder, Hunter syndrome, Menkes syndrome, Rett syndrome, Tay-Sachs disease, Niemann-Pick, Hurler syndrome, Hallerporden-Spatz disease, metachromatic leukodystrophy, or Krabbe disease, etc., but is not limited thereto.

The term “prevention”, used herein, refers to all actions that block an occurrence of a disease in advance, suppress the disease, or delay progression of the disease.

The term “treatment”, used herein, means both therapeutic treatment and prophylactic or preventive methods. In addition, the term refers to any action that improves or beneficially changes symptoms of a disease.

The pharmaceutical composition may be provided in an injectable form. Therefore, the pharmaceutical composition may include a pharmaceutically acceptable carrier, etc., and particularly, may be blended to be administered by topical, oral, parenteral, intranasal, intravenous, intramuscular, subcutaneous, intraocular, or transdermal routes. Preferably, the pharmaceutical composition may contain basic pharmaceutically acceptable excipients, particularly in injectable formulations for direct injection into the nervous system of a patient. These injectable formulations may be sterile, isotonic solutions, or dry, particularly lyophilized compositions, which allow the injectable solutions to be particularly constituted of sterile water or physiological saline to be added thereto depending on a case.

The pharmaceutically acceptable carriers are commonly used in formulation, and include lactose, dextrose, sucrose, sorbitol, mannitol, starch, acacia gum, calcium phosphate, gelatin, alginate, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, and the like, but are not limited thereto.

The pharmaceutical composition may further include a lubricant, a wetting agent, a sweetening agent, a flavoring agent, an emulsifying agent, a suspending agent, a preservative, and the like, in addition to the above components.

The pharmaceutical composition may be administered orally or parenterally. When the pharmaceutical composition is administered parenterally, the pharmaceutical composition may be administered by intravenous injection, subcutaneous injection, intramuscular injection, intraperitoneal injection, endothelial administration, topical administration, intranasal administration, intrapulmonary administration, and intrarectal administration, and most preferably, may be administered intravenously. When the pharmaceutical composition is administered orally, the pharmaceutical composition may be formulated to be protected from degradation in the stomach, or the active agent may be coated. In addition, the pharmaceutical composition may be administered by any device capable of transporting an active substance to a target cell.

A suitable dosage of the pharmaceutical composition may be determined in various ways depending on factors such as formulation method, administration method, age, weight, sex, and disease state of the patient, food, administration time, route of administration, excretion rate and response sensitivity.

The pharmaceutical composition may be for gene therapy.

The term “gene therapy”, used herein, refers to a therapeutic method that uses genes to treat or prevent a disease.

The gene therapy may include a method of inserting a new gene into cells of a subject, removing a gene that is malfunctioning, or replacing a mutated gene with a normal gene.

Among the terms or elements mentioned in the description of the pharmaceutical composition, the same ones as mentioned in the description of the composition for gene delivery are understood to be the same as mentioned in the description of the composition for gene delivery.

Still another aspect provides a method of preparing the virus-dendrimer-peptide complex for delivering a gene to brain tissue, including connecting a dendrimer and a cerebrovascular cell-targeting peptide to a surface of a recombinant viral vector.

Still another aspect provides a use of the virus-dendrimer-peptide complex including the recombinant viral vector, and the dendrimer and the cerebrovascular cell-targeting peptide connected to a surface of the recombinant virus, for delivering a gene to brain tissue.

Among the terms or elements mentioned in the description of the preparation method and use, the same ones as mentioned in the description of the composition for gene delivery and the pharmaceutical composition are understood to be the same as mentioned in the description of the composition for gene delivery and the pharmaceutical composition.

### Advantageous Effects of Disclosure

According to a composition according to an aspect, target genetic material may be delivered to cerebrovascular cells, or the target genetic material may be delivered to brain tissue across the brain surface barrier, blood-brain barrier, or the blood-cerebrospinal fluid barrier. Due to this, brain diseases may be prevented or treated.

### BRIEF DESCRIPTION OF DRAWINGS

FIG. 1 is a schematic diagram showing a process for selecting phages having vascular endothelial cell-targeting peptides by using a CX7C M13 phage library.

FIG. 2 is a graph showing results of an Phage ELISA analysis for analyzing targeting ability of the phages, which have vascular endothelial cell-targeting peptides, for vascular endothelial cells.

FIG. 3 is a diagram showing results of analyzing sequence patterns to derive a dominant motif related to a property of targeting vascular endothelial cells from the peptide sequences of the phages based on the results of FIG. 2 above.

FIG. 4 is a diagram showing two types of PAMAM dendrimers, G2 and G5, which have positively charged surfaces with NH₂ functional groups bound to their surfaces.

FIG. 5 is a diagram showing an example of the dendrimer-peptide complex in which a PAMAM dendrimer and a cerebrovascular cell-targeting peptide are conjugated.

FIG. 6 is a schematic diagram showing an example of a process for preparing a rAAV-dendrimer-peptide complex in which a surface of an rAAV vector is coated with the dendrimer-peptide complex.

FIG. 7A is a photograph of green fluorescent protein (GFP) expression patterns showing results of analyzing targeting abilities of rAAV-dendrimer-peptide complexes (rAAV2/6-G2P1 and rAAV2/6-G2P3) for vascular endothelial cells.

FIG. 7B is a graph showing results of quantitatively analyzing the GFP expression patterns of FIG. 7A.

FIG. 8A is a photograph of GFP expression patterns showing results of analyzing abilities of rAAV-dendrimer-peptide complexes (rAAV2/9-G2P1 and rAAV2/9-G2P3) to deliver a gene to cerebrovascular (capillary and artery) endothelial cells.

FIG. 8B is a graph showing results of quantitatively analyzing fluorescence intensity of GFP expression of FIG. 8A.

FIG. 9A is a photograph of GFP expression patterns showing results of analyzing an ability of an rAAV-dendrimer-peptide complex (rAAV2/9-G2P3) to deliver a gene to the cortical tissue located inside the arachnoid barrier constituting the brain surface barrier.

FIG. 9B is a photograph of GFP expression patterns showing results of analyzing an ability of an rAAV-dendrimer-peptide complex (rAAV2/9-G2P3) to deliver a gene to the cerebral cortical tissue located inside the blood-brain barrier.

FIG. 9C is a photograph of GFP expression patterns showing results of analyzing an ability of an rAAV-dendrimer-peptide complex (rAAV2/9-G2P3) to deliver a gene to the hippocampal tissue located inside the blood-cerebrospinal fluid barrier.

FIG. 10 is a diagram showing positions of (A) the cortical tissue located inside the arachnoid barrier constituting the brain surface barrier, (B) the cerebral cortical tissue located inside the blood-brain barrier, and (C) the hippocampal tissue located inside the blood-cerebrospinal fluid barrier.

### MODE OF DISCLOSURE

Hereinafter, the present disclosure will be described in more detail through examples. However, these examples are intended to illustrate at least one embodiment, and the scope of the present disclosure is not limited to these examples.

### Examples 1 to 4. Preparation of Recombinant Viral Vectors

### 1-1. Construction of Viral Vectors

The constructed plasmids (pAAV-CMV-eGFP and pAAV-CMV-Cre) include 5′-inverted terminal repeats (ITR), cytomegalovirus (CMV) enhancer promoters, TRANS (transportation) genes (eGFP or Cre), regulatory elements after transcription (WPRE: woodchuck hepatitis virus posttranscriptional regulatory element), bovine growth hormone polyadenylation signal (bGHpA), 3′-ITR, and resistance region to an antibiotic ampicillin (Amp), etc.

1-2. Preparation of Recombinant Adeno-Associated Virus (rAAV) Vectors

In this example, a triple plasmid transfection method was performed to prepare a recombinant AAV vector. The three types of plasmids are an adenovirus (Ad) helper plasmid (pAdΔF6), a chimeric transplasmid (crosspackaged pseudotyped vectors) containing the AAV2 rep gene fused to a capsid gene of the desired AAV serotype, and an ITR-positive rAAV vector plasmid. The triple plasmid transfection method may be performed specifically according to methods known in the art.

Specifically, in order to prepare rAAV2-CMV-eGFP viral vectors, Ad helper plasmids (pAdΔF6) (SEQ ID NO: 35), chimeric transplasmids (SEQ ID NO: 36) containing the AAV2 rep gene (pDG), and pAAV-CMV-eGFP plasmids (SEQ ID NO: 37) were cotransfected into U293 cells.

In addition, in order to prepare rAAV2/6-CMV-eGFP viral vectors, Ad helper plasmids (pAdΔF6) (SEQ ID NO: 35), chimeric transplasmids containing the AAV2 rep gene (pDP6), and pAAV-CMV-eGFP plasmids (SEQ ID NO: 37) were cotransfected into U293 cells.

In addition, in order to prepare rAAV2/9-CMV-eGFP or rAAV2/9-CMV-Cre viral vectors, Ad helper plasmids (pAdΔF6) (SEQ ID NO: 35), chimeric transplasmids (SEQ ID NO: 38) including the AAV2 rep gene (pDP9), and pAAV-CMV-eGFP plasmids (SEQ ID NO: 37) or pAAV-CMV-Cre plasmids (SEQ ID NO: 39) were cotransfected into U293 cells.

After culturing the transfected cells for 2 days to 3 days, the cells were harvested and viral vectors were purified by using standard cesium sedimentation. A titer of the rAAV viral vector particles was determined by using qPCR for the untranslated regions of the DNA-encoded transcripts in each AAV vector. For qPCR, a bGHpA primer (SEQ ID NO: 3: forward, 5′TCT AGT TGC CAG CCA TCT GTT GT 3′; SEQ ID NO: 4: reverse, 5′TGG GAG TGG CAC CTT CA3′), a Cre primer (SEQ ID NO: 5: forward, 5′AGA GGA AAG TCT CCA ACC TG 3′; SEQ ID NO: 6: reverse, 5′ACA CAG ACA GGA GCA TCT TC 3′), and an eGFP primer (SEQ ID NO: 7: forward, 5′GCC ACA ACG TCT ATA TCA TGG 3′; SEQ ID NO: 8: reverse, 5′ GGT GTT CTG CTG GTA GTG GT 3′) were used. In addition, real-time PCR using SYBR Green was performed by an iQ-Cycler (Bio Rad, Germany) for 40 cycles (1 cycle: 95° C. for 30 sec, 58° C. for 30 sec, and 72° C. for 30 sec).

As a result, recombinant viral vectors rAAV2-CMV-eGFP (Example 1), rAAV2/6-CMV-eGFP (Example 2), rAAV2/9-CMV-eGFP (Example 3), and rAAV2/9-CMV-Cre (Example 4) were obtained.

### Examples 5 to 6. Preparation of Cerebrovascular Cell-Targeting Peptides

### 5-1. Discovery of Cerebrovascular Cell-Targeting Phages

In this example, an M13 phage library was used to discover phages that target cerebrovascular cells.

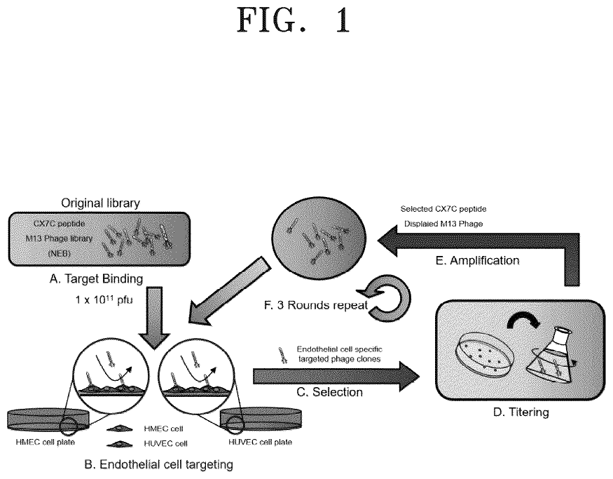

Specifically, as shown in FIG. 1, 2×10¹¹ plaque forming units (pfu) of the CX7C M13KE phage library was incubated for 120 minutes at room temperature with human mammary epithelial cells (HMECs) or human umbilical vein endothelial cells (HUVECs) of a concentration of 1×10⁷ cell/plate, along with 1 ml of 1% BSA/DMEM. Thereafter, the culture plate was washed to remove phages that did not bind to vascular endothelial cells, and phages bound to vascular endothelial cells were selected. The selected bound phages were eluted and then amplified in E. coli. Specifically, each of 488 single blue (positive) plaques was selected from the phages eluted from the LB/IPTG/X gal plate, and the selected plaques were inoculated into a small amount of bacterial culture of E. coli (A600 nm OD 0.5) and incubated at 37° C. for 4.5 hours with shaking. After purifying the culture medium, 1 μl of the selected phage was PCR analyzed by using M13 phage PCR primers (SEQ ID NO: 9: forward, 5′TTA TTC GCA ATT CCT TTA GTG G 3′; SEQ ID NO: 10: reverse, 5′CCC TCA TAG TTA GCG TAA CG 3′). In addition, amino acid sequences of the discovered phages were analyzed.

As a result, as shown in Table 1 below, a total of 12 amino acid sequences of peptides of vascular endothelial cell-targeting phages were obtained. Phages U1 to U5 in Table 1 below were selected after panning in HUVEC, and phages M1 to M7 were selected after panning in HMEC.

TABLE 1

| | Name | Sequence | |
|---|---|---|---|
| 1 | U4: | SEQ ID NO: 11: NCLANPC | SEQ ID NO: 23: |
| | HUVEC3R085 | | TGTTGAAATTGTTTAGCA |
| | | | AATCCCTGC |
| 2 | U5: | SEQ ID NO: 12: CSVTKSTYC | SEQ ID NO: 24: |
| | HUVEC3R076 | | TGTTCTGTGACGAAGTCG |
| | | | ACGTATTGC |
| 3 | U1: | SEQ ID NO: 13: CRSVSNNNC | SEQ ID NO: 25: |
| | HUVEC2R061 | | TGTCGTAGTGTGTCGAAT |
| | | | AATAATTGC |
| 4 | U2: | SEQ ID NO: 14: CSHMNESMC | SEQ ID NO: 26: |
| | HUVEC2R064 | | TGTTCTCATATGAATGAG |
| | | | TCGATGTGC |
| 5 | U3 | SEQ ID NO: 15: CRFHMRETC | SEQ ID NO: 27: |
| | HUVEC3R011 | | TGTAGATTTCATATGAGG |
| | | | GAGACTTGC |
| 6 | M1: | SEQ ID NO: 16: CLYLGAEMC | SEQ ID NO: 28: |
| | HMEC2R031 | | TGTCTGTATTTGGGTGCT |
| | | | GAGATGTGC |
| 7 | M3: | SEQ ID NO: 17: CTITGQGAC | SEQ ID NO: 29: |
| | HMEC3R043 | | TGTACGATTACTGGTCAG |
| | | | GGTGCTTGC |
| 8 | M2: | SEQ ID NO: 18: CTQSGVRNC | SEQ ID NO: 30: |
| | HMEC3R042 | | TGTACGCAGTCGGGGGT |
| | | | TAGGAATTGC |
| 9 | M7: | SEQ ID NO: 19: CTQSGVRNC | SEQ ID NO: 31: |
| | HMEC3R046 | | TGTACGCAGTCGGGGGT |
| | | | TAGGAATTGC |
| 10 | M4: | SEQ ID NO: 20: VGGWNC | SEQ ID NO: 32: |
| | HMEC3R115 | | TGTAACTGAGTCGGGGG |
| | | | TTGGAATTGC |
| 11 | M5: | SEQ ID NO: 21: CILSNNFFC | SEQ ID NO: 33: |
| | HMEC3R124 | | TGTATCCTTAGTAATAATT |
| | | | TCTTTTGC |
| 12 | M6: | SEQ ID NO: 22: CMVSMILRC | SEQ ID NO: 34: |
| | HMEC3R125 | | TGTATGGTAAGTATGATC |
| | | | CTACGGTGC |

### 5-2. Securing Optimized Amino Acid Sequences of Cerebrovascular Cell-Targeting Peptides

In this example, targeting abilities of the selected phages in Table 1 for vascular endothelial cells were analyzed by a Phage ELISA analysis, in order to secure optimized amino acid sequences of cerebrovascular cell-targeting peptides. In addition, based on the results, the optimized amino acid sequences of cerebrovascular cell-targeting peptides were derived by analyzing the dominant motif from the amino acid sequences of the selected phages in Table 1.

Specifically, each of the 10 phage clones in Table 1 (4 phage clones screened in HUVEC and 6 phage clones screened in HMEC) was incubated with vascular endothelial cells (HUVEC or HMEC) at room temperature for 120 minutes to confirm the binding of phage clones to the vascular endothelial cells, and non-binding phage clones were removed. Phage clones bound to vascular endothelial cells were incubated overnight at 4° C. with horseradish peroxidase (HRP)-conjugated M13 PVIII antibodies, followed by absorbance analysis at 450 nm by using 3,3′,5,5′-tetramethylbenzidine (TMB) substrate.

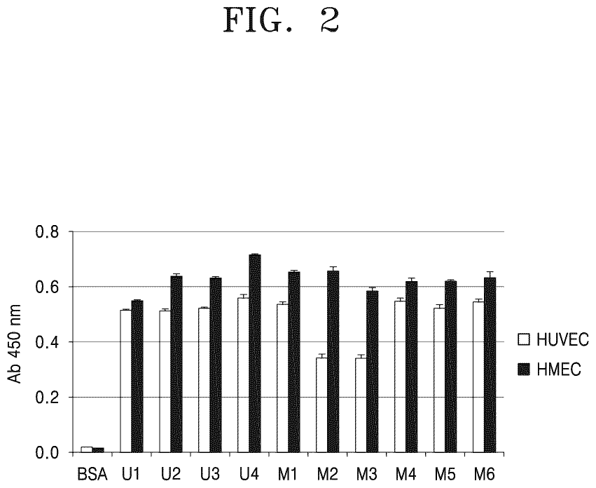

As a result, as shown in FIG. 2, it was confirmed that U1 to U4, M1, and M4 to M6 phages could bind to both HUVEC and HMEC with high affinity, whereas M2 and M3 phages could bind with high affinity to only HMEC.

In addition, based on the results of FIG. 2, sequence patterns of phage sequences in Table 1 were analyzed by using WebLogo3, and dominant motifs related to the property of targeting vascular endothelial cells were derived from the phage sequences.

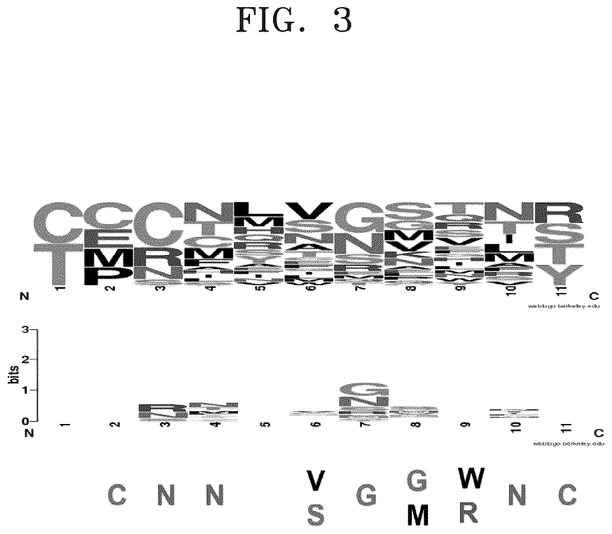

As a result, as shown in FIG. 3, the first four amino acids in the sequence, asparagine (Asn, N), N, serine (Ser, S), and glycine (Gly, G), and the last amino acid in the sequence, N, were confirmed to be dominant motifs. In addition, according to alignment analysis results for the phage sequences in Table 1 and phage ELISA analysis results in FIG. 2, the fifth and sixth amino acids in the sequence were determined by using the Clustal W program.

Finally, as shown in Table 2 below, peptide P3 having an optimized amino acid sequence capable of targeting vascular endothelial cells was obtained. In addition, a peptide P1 having an amino acid sequence of a vascular endothelial cell-targeting peptide inserted on the A589 surface of an AAV9 capsid obtained by AAV library screening was obtained.

TABLE 2

| | Name | Sequence |
|---|---|---|
| Example 5 | P1 | SEQ ID NO: 1: CSLRSPPS |
| Example 6 | P3 | SEQ ID NO: 2: CNNSGMRN |

Finally, through this example, peptide P3 (Example 6) having an optimized amino acid sequence capable of targeting vascular endothelial cells was obtained. In particular, the peptide P3 is a cerebrovascular cell-targeting peptide with remarkably excellent ability to target cerebrovascular endothelial cells, which was demonstrated in the following experimental examples.

### Examples 7 to 10. Preparation of Dendrimer-Peptide Complex in which Polyamidoamine (PAMAM) Dendrimer and Cerebrovascular Cell-Targeting Peptide are Conjugated

In this example, a dendrimer-peptide complex in which a PAMAM dendrimer and a cerebrovascular cell-targeting peptide were conjugated was prepared.

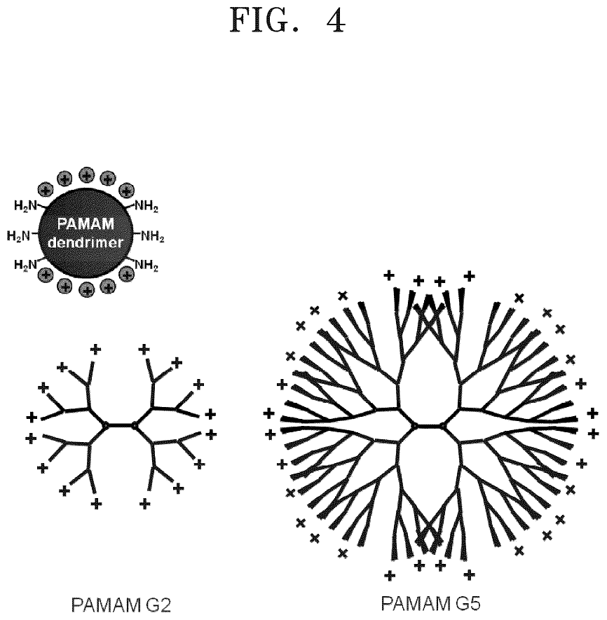

Specifically, as shown in FIG. 4 and Table 3, two types of PAMAM dendrimers, G2 and G5, which have positively charged surfaces by having NH₂ functional groups bound to the surface, were purchased from Dendritic Nanotech Inc. (Michigan, USA).

TABLE 3

| | | | Hydro dynamic | No. of NH₂ |
|---|---|---|---|---|
| | MW | Molecular formula | diameter (nm) | surface groups |
| G2 | 3,348 | C₁₄₄H₂₉₂N₅₈O₂₈S₂ | 2.9 | 16 |
| G5 | 28,918 | C₁₂₆₄H₂₅₈₂N₅₀₈O₂₅₂S₂ | 5.4 | 128 |

Dendrimer-peptide complexes were prepared by conjugating P1 or P3 of Table 2 to surfaces of G2 and G5, respectively. Specifically, 1 μmol of G2 (MW: 3,284 Da) was dissolved in dimethyl sulfoxide (DMSO), and then incubated with 4 μmol of NHS-PEG-OPSS linker (2 kDa) at 37° C. for 3 hours. The reaction mixture was loaded onto a cation exchange column (Macro Prep High S, BioRad) and fractionated by using a 20 mM HEPES (pH 7.4) solution with a NaCl salt gradient of 0.6 M to 3 M. Then, the purified product was filtered with a centrifugal filter device (Amicon Ultra 3K) and G2 content was determined by a TNBS analysis.

To prepare dendrimer-peptide complexes G2P1 and G2P3, peptide P1 or P3 (1.98 μmol) dissolved in 75 μl of 30% acetonitrile, 70% H₂O, 0.1% trifluoroacetic acid (TFA) solution, and 3.6 ml of G2 (0.79 μmol) conjugated with PEG-OPSS linkers dissolved in an HBS solution were mixed and incubated at room temperature. After the incubation, the mixture was loaded onto a cation exchange column with a 20 mM HEPES solution containing 10% acetonitrile solution (pH 7.4) and having a NaCl salt gradient of 0.6 M to 3 M. Then, the purified product was filtered with a centrifugal filter device, and G2 content was determined by a TNBS analysis. An amount of P1 or P3 was calculated by using the extinction coefficient at 280 nm.

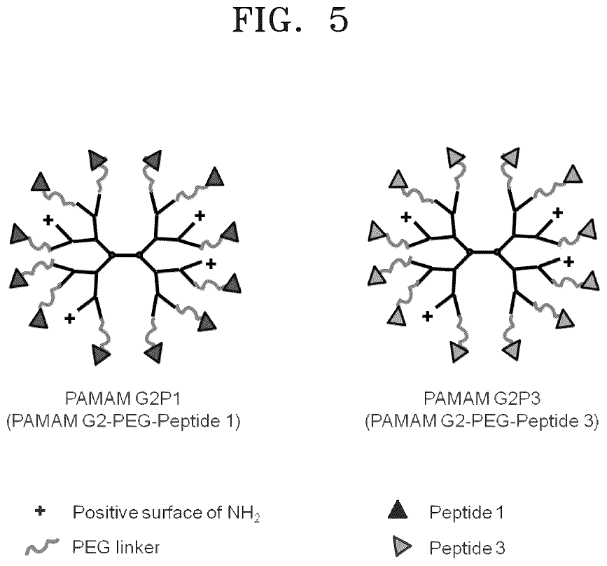

As a result, as shown in FIG. 5 and Table 4, dendrimer-peptide complexes were obtained.

TABLE 4

| | Dendrimer-peptide complex | Construction |
|---|---|---|
| Example 7 | G2P1 | PAMAM G2-PEG-P1 |
| Example 8 | G2P3 | PAMAM G2-PEG-P3 |
| Example 9 | G5P1 | PAMAM G5-PEG-P1 |
| Example 10 | G5P3 | PAMAM G5-PEG-P3 |

### Examples 11 to 26. Preparation of Virus-Dendrimer-Peptide Complexes in which Dendrimer-Peptide Complexes are Coated on Surface of Recombinant Viral Vectors

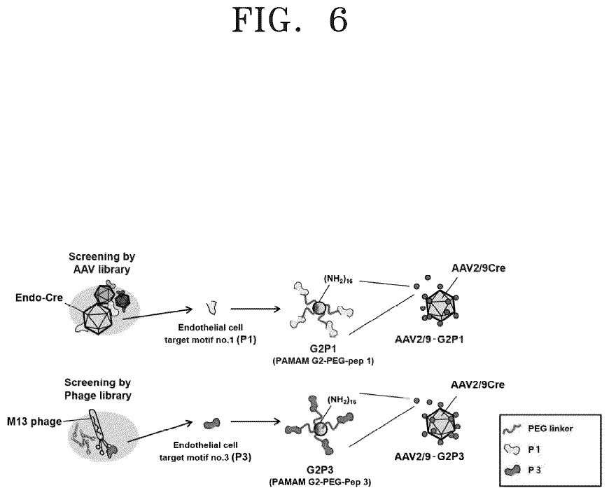

In this example, as shown in FIG. 6, rAAV-dendrimer-peptide complexes (virus-dendrimer-peptide complex) were prepared, in which the dendrimer-peptide complexes of Table 4 are coated on a surface of each of the recombinant viral vectors of Examples 1 to 4.

Specifically, after diluting each of the recombinant viral vectors of Examples 1 to 4 and each of the dendrimer-peptide complexes of Table 4 with an Opti MEM (Invitrogen, Germany) solution, 25 μl of a diluted solution of the recombinant viral vectors (10¹⁰ GC/ml to 10²⁰ GC/ml, specifically, 1.0E+13 GC/ml)) was added to 25 μl of a diluted solution of the dendrimer-peptide complexes (10¹⁵ NP/ml to 10²⁵ NP/ml, specifically, 1.39E+21 NP/ml), gently mixed, and incubated at room temperature for about 10 minutes to about 30 minutes.

As a result, as shown in Table 5 below, a total of 16 types of rAAV-dendrimer-peptide complexes (virus-dendrimer-peptide complexes) were obtained.

TABLE 5

| | | | Virus- |
|---|---|---|---|
| | | Dendrimer- | dendrimer- |
| | Recombinant | peptide | peptide |
| | viral vector | complex | complex |
| Example | rAAV2(rAAV2- | G2P1 | rAAV2-G2P1 |
| 11 | CMV-eGFP) | | |
| Example | rAAV2(rAAV2- | G2P3 | rAAV2-G2P3 |
| 12 | CMV-eGFP) | | |
| Example | rAAV2(rAAV2- | G5P1 | rAAV2-G5P1 |
| 13 | CMV-eGFP) | | |
| Example | rAAV2(rAAV2- | G5P3 | rAAV2-G5P3 |
| 14 | CMV-eGFP) | | |
| Example | rAAV2/6(rAAV2/6- | G2P1 | rAAV2/6-G2P1 |
| 15 | CMV-eGFP) | | |
| Example | rAAV2/6(rAAV2/6- | G2P3 | rAAV2/6-G2P3 |
| 16 | CMV-eGFP) | | |
| Example | rAAV2/6(rAAV2/6- | G5P1 | rAAV2/6-G5P1 |
| 17 | CMV-eGFP) | | |
| Example | rAAV2/6(rAAV2/6- | G5P3 | rAAV2/6-G5P3 |
| 18 | CMV-eGFP) | | |
| Example | rAAV2/9(rAAV2/9- | G2P1 | rAAV2/9-G2P1 |
| 19 | CMV-eGFP) | | |
| Example | rAAV2/9(rAAV2/9- | G2P3 | rAAV2/9-G2P3 |
| 20 | CMV-eGFP) | | |
| Example | rAAV2/9(rAAV2/9- | G5P1 | rAAV2/9-G5P1 |
| 21 | CMV-eGFP) | | |
| Example | rAAV2/9(rAAV2/9- | G5P3 | rAAV2/9-G5P3 |
| 22 | CMV-eGFP) | | |
| Example | rAAV2/9Cre(rAAV2/9- | G2 | rAAV2/9Cre-G2 |
| 23 | CMV-Cre) | | |
| Example | rAAV2/9Cre(rAAV2/9- | G2P3 | rAAV2/9Cre-G2P3 |
| 24 | CMV-Cre) | | |
| Example | rAAV2/9Cre(rAAV2/9- | G5 | rAAV2/9Cre-G5 |
| 25 | CMV-Cre) | | |
| Example | rAAV2/9Cre(rAAV2/9- | G5P3 | rAAV2/9Cre-G5P3 |
| 26 | CMV-Cre) | | |

### Experimental Example 1. Confirmation of Targeting Ability of Virus-Dendrimer-Peptide Complex for Vascular Endothelial Cells

In this experimental example, targeting abilities of the prepared rAAV-dendrimer-peptide complexes (virus-dendrimer-peptide complex) for vascular endothelial cells were evaluated in vitro.

Specifically, rAAV-dendrimer-peptide complexes (rAAV2/6-G2P1 or rAAV2/6-G2P3), in which G2P1 or G2P3 is surface coated on rAAV2/6-CMV-eGFP viral vector particles, were transfected into HMEC at a concentration of 5.0E+05 vp/cell for 72 hours at 37° C. As control groups, rAAV2/6-CMV-eGFP vectors alone, rAAV2/6-CMV-eGFP vectors surface coated with G2, and rAAV2/6-CMV-eGFP vectors surface coated with G5 were transfected into HMECs. Then, intensity of green fluorescent protein (GFP) expression per total red fluorescent protein (RFP) area was analyzed.

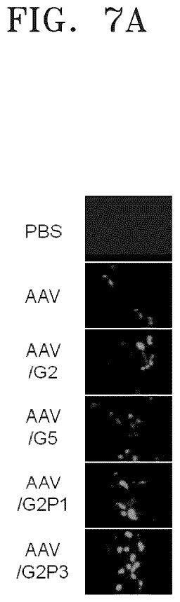

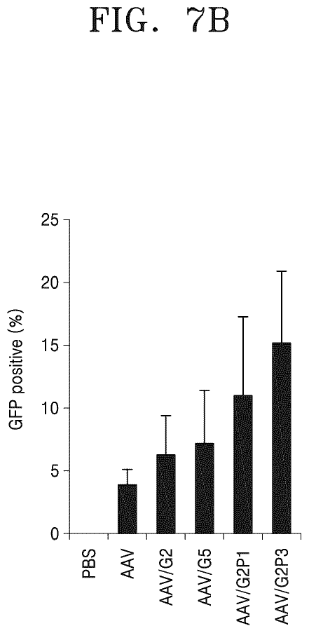

As a result, as shown in FIGS. 7A and 7B, it was confirmed that the rAAV-dendrimer-peptide complexes (rAAV2/6-G2P1 and rAAV2/6-G2P3) had significantly superior ability to target vascular endothelial cells compared to the control groups. In particular, the ability of the rAAV-dendrimer-peptide complexes (rAAV2/6-G2P3) containing P3 peptides to target vascular endothelial cells was found to be significantly superior to that of the rAAV-dendrimer-peptide complexes (rAAV2/6-G2P1) containing P1 peptides.

These results support that the virus-dendrimer-peptide complex is capable of targeting cerebrovascular endothelial cells, which was specifically demonstrated in the following experimental example.

### Experimental Example 2. Confirmation of Ability of Virus-Dendrimer-Peptide Complexes to Target Cerebrovascular Endothelial Cells and Transfer Gene to Brain Tissue

In this experimental example, the ability of the prepared rAAV-dendrimer-peptide complexes (virus-dendrimer-peptide complex) to target vascular endothelial cells, and to transfer genes to brain tissue was evaluated in vivo.

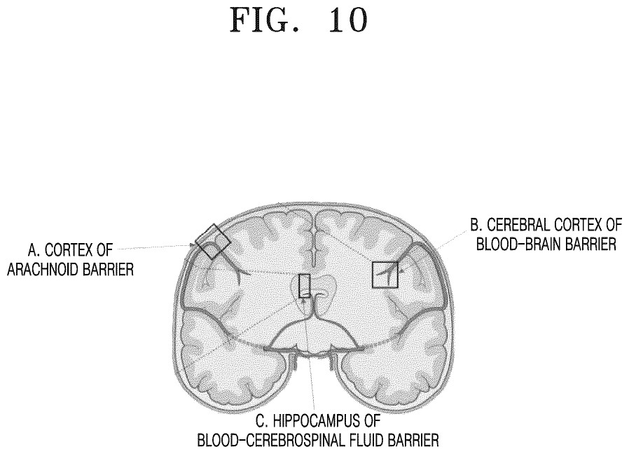

Specifically, Dtamato mice (10 weeks old) were used as animal models, and rAAV-dendrimer-peptide complexes (rAAV2/9-G2P1 or rAAV2/9-G2P3), in which G2P1 or G2P3 is surface coated on rAAV2/9-CMV-eGFP viral vector particles were intravenously (IV) injected into the animal models. 48 hours after the injection, each organ (brain, heart, liver, kidney, hind limbs, and spleen) of the animal models was extracted while being perfused, and each of the extracted organs was observed by taking photos under a confocal microscope. Specifically, in cerebrovascular (capillary and artery) tissues, cortex tissues located inside the arachnoid barrier constituting the brain surface barrier (see A in FIG. 10), cerebral cortex tissues located inside the blood-brain barrier (see B in FIG. 10), and hippocampus tissues located inside the blood-cerebrospinal fluid barrier (see C in FIG. 10), the expression of GFP, a TRANS gene was observed by taking photos under a confocal microscope.

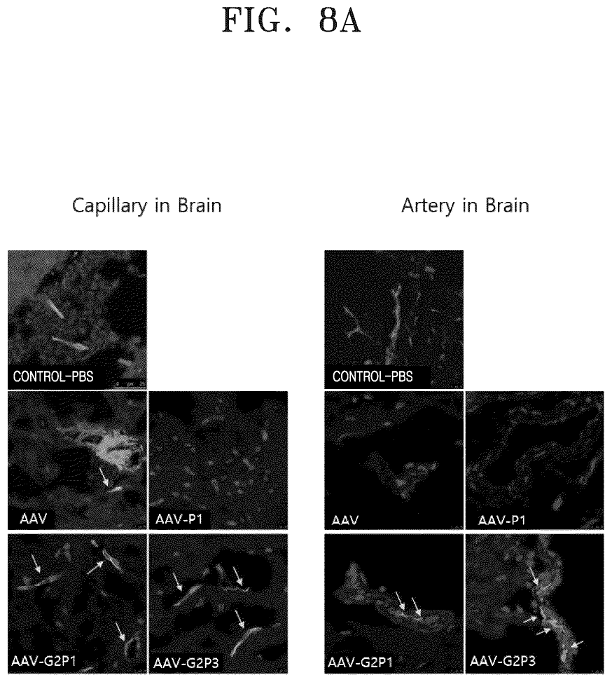

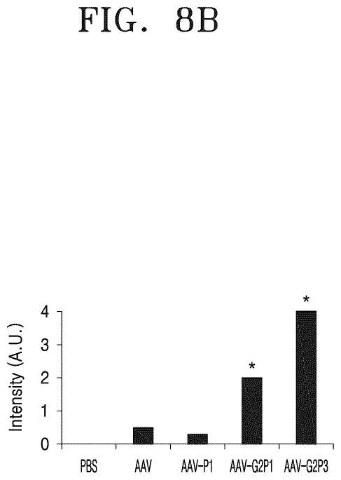

As a result, as shown in FIGS. 8A and 8B, the rAAV-dendrimer-peptide complexes (rAAV2/9-G2P1 and rAAV2/9-G2P3) were found to have remarkably excellent ability to target cerebrovascular (capillary and artery) endothelial cells, and to deliver genes to cerebrovascular endothelial cells. In particular, the ability of the rAAV-dendrimer-peptide complex (rAAV2/9-G2P3) containing a P3 peptide to target cerebrovascular endothelial cells and to transfer a gene to a cerebrovascular endothelial cell was confirmed to be remarkably superior compared to that of the rAAV-dendrimer-peptide complex (rAAV2/9-G2P1) containing a P1 peptide.

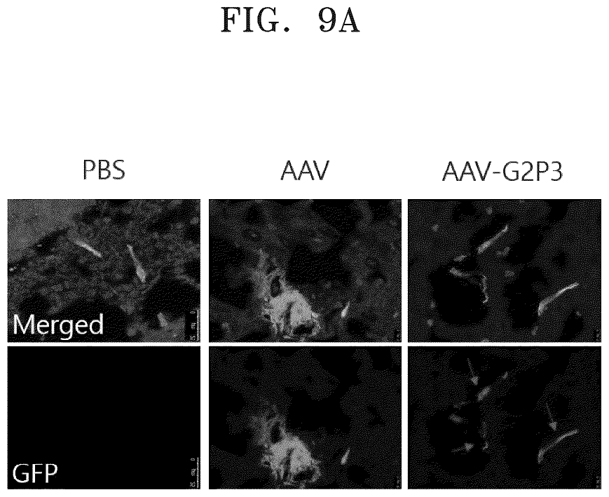

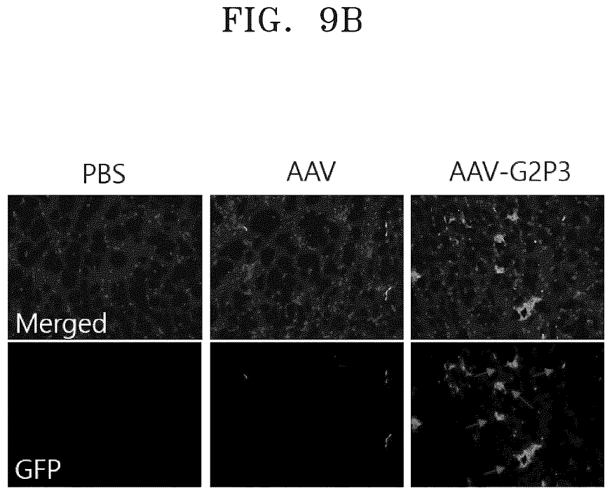

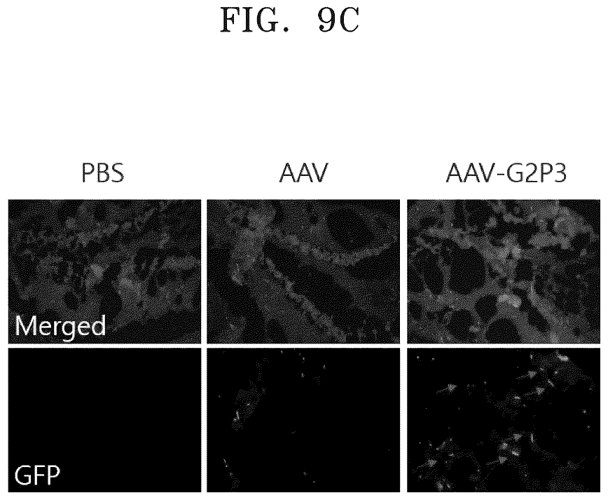

In addition, as shown in FIG. 9A, it was confirmed that the rAAV-dendrimer-peptide complex (rAAV2/9-G2P3) may deliver a gene to cortical tissue located inside the arachnoid barrier constituting the brain surface barrier. In addition, as shown in FIG. 9B, it was confirmed that the rAAV-dendrimer-peptide complex (rAAV2/9-G2P3) may deliver a gene to cerebral cortical tissue located inside the blood-brain barrier. In addition, as shown in FIG. 9C, it was confirmed that the rAAV-dendrimer-peptide complex (rAAV2/9-G2P3) may deliver a gene to the hippocampal tissue located inside the blood-cerebrospinal fluid barrier. On the other hand, when the AAV2/9 vectors (AAV2/9) were injected alone, it was confirmed that genes could be sufficiently transferred to the cortical tissue located inside the arachnoid barrier constituting the brain surface barrier (FIG. 9A), but an amount of transferred genes to the cortical tissue located inside the blood-brain barrier and the hippocampal tissue located inside the blood-cerebrospinal fluid barrier were significantly reduced (FIGS. 9B and 9C).

Through this experimental example, it was confirmed that the virus-dendrimer-peptide complex containing a P3 peptide may deliver a target gene to a cerebrovascular endothelial cell. In addition, through this experimental example, it was confirmed that the virus-dendrimer-peptide complex containing a P3 peptide may pass through the brain surface barrier, the blood-brain barrier, and the blood-cerebrospinal fluid barrier, and thereby, a target gene may be delivered to brain tissues such as the brain cortex and the hippocampus, etc. inside the brain barrier. From this, it was found that the virus-dendrimer-peptide complex containing a P3 peptide is capable of delivering a target gene not only to cerebrovascular endothelial cells, but also to cranial nerve cells existing in the brain cortex and hippocampus, etc. inside the brain barrier.

From the above description, those skilled in the art to which this application belongs will be able to understand that this application may be implemented in other specific forms without changing its technical idea or essential features. Therefore, it should be understood that the above examples are not limitative, but illustrative in all aspects. Scope of the present application should be construed as including meanings and scope of the claims to be followed, and all changes or modifications derived from equivalent concepts thereof, rather than the detailed description above.

1. A composition for gene delivery to brain tissue comprising a virus-dendrimer-peptide complex comprising: a recombinant viral vector; and a dendrimer and a cerebrovascular cell-targeting peptide linked to a surface of the recombinant viral vector.

2. The composition of claim 1, wherein the dendrimer has a positive charge at the end of the outermost surface.

3. The composition of claim 2, wherein the dendrimer is a dendrimer of any one type selected from the group consisting of a polyamidoamine dendrimer, polylysine dendrimer, polyimine dendrimer, polypropyleneimine dendrimer, polyester dendrimer, polyether dendrimer, polyglutamic acid dendrimer, polyaspartic acid dendrimer, polyglycerol dendrimer, and polymelamine dendrimer, or a dendrimer composed of a copolymer of two or more types selected from the group.

4. The composition of claim 1, wherein the cerebrovascular cell-targeting peptide is a peptide comprising an amino acid sequence of SEQ ID NO: 1 or SEQ ID NO: 2.

5. The composition of claim 1, wherein one end of the dendrimer is connected to the cerebrovascular cell-targeting peptide by a linker.

6. The composition of claim 1, wherein the brain tissue is cerebrovascular cells or cranial nerve cells.

7. The composition of claim 1, wherein the recombinant viral vector comprises a target gene.

8. The composition of claim 1, wherein the virus-dendrimer-peptide complex passes through the blood-brain barrier (BBB).

9. A pharmaceutical composition for preventing or treating a brain disease comprising the composition for gene delivery of claim 1 as an active ingredient.

10. The pharmaceutical composition of claim 9, wherein the pharmaceutical composition is administered intravenously.

11. The pharmaceutical composition of claim 9, wherein the pharmaceutical composition is for gene therapy.

12. A method of delivering genes into a subject's brain tissue, the method comprising:
administering a composition for gene delivery to brain tissue comprising a virus-dendrimer-peptide complex to a subject,
wherein the virus-dendrimer-peptide complex comprises a recombinant viral vector; and a dendrimer and a cerebrovascular cell-targeting peptide linked to a surface of the recombinant viral vector, and
wherein the recombinant viral vector comprises a target gene to be delivered to brain tissue.

13. The method of claim 12, wherein the cerebrovascular cell-targeting peptide is a peptide comprising an amino acid sequence of SEQ ID NO: 1 or SEQ ID NO: 2.

14. The method of claim 12, wherein one end of the dendrimer is connected to the cerebrovascular cell-targeting peptide by a linker.

15. The method of claim 12, wherein the brain tissue is cerebrovascular cells or cranial nerve cells.

16. The method of claim 12, wherein the virus-dendrimer-peptide complex passes through the blood-brain barrier (BBB).
